(19) United States
(12) Patent Application Publication
(10) Pub. No.: US 2025/0213461 A1
(43) Pub. Date: Jul. 3, 2025

(54) A MESENCHYMAL STEM CELLS FORMULATION FOR COSMETIC USE

(71) Applicant: BRIGHT CELL, INC., Irvine, CA (US)

(72) Inventors: Wenbin LIAO, Irvine, CA (US); Linan LIU, Irvine, CA (US)

(21) Appl. No.: 18/853,149

(22) PCT Filed: Mar. 30, 2023
(86) PCT No.: PCT/US2023/016909

§ 371 Date: Oct. 1, 2024

Related U.S. Application Data
(60) Provisional application No. 63/326,594, filed on Apr. 1, 2022.

(51) Int. Cl. A61K 8/98; A61K 8/44; A61K 8/64; A61K 8/73; A61Q 19/08

(52) U.S. Cl. CPC A61K 8/981; A61K 8/44; A61K 8/64; A61K 8/73; A61Q 19/08

(57) ABSTRACT

The invention provides compositions of a mesenchymal stem cells (MSC) formulation, a characterization method to define the potency of MSC for cosmetic purposes and use of the MSC product to ameliorate the skin wrinkles, reduce the signs of aging attenuate skin scar or treat hair loss. Practicing these methods and MSC formulation application will greatly improve the quality and appearance of the skin.

This application claims priority to U.S. Provisional Patent Application No. 63/326,594, filed on Apr. 1, 2022, which is incorporated by reference for all purposes as if fully set forth herein.

### TECHNICAL FIELD

The present invention relates to compositions of a mesenchymal stem cells (MSC) formulation, a characterization method to identify the MSC potency and use of the MSC formulation in cosmetics.

### BACKGROUND

Skin is the largest and most visible organ of the body. It comprises three layers, epidermis, dermis, and hypodermis. Like all other tissues, the skin undergoes degenerative processes during aging, and the effects of aging on the skin are most visible. Changes in aged skin include structural, biochemical, and functional changes. Skin aging is a complex biological process influenced by the combination of intrinsic and extrinsic factors, which lead to structural and physiological changes in each skin layer, resulting in changes in skin appearance. Intrinsic aging is generally under genetic and hormonal influence and extrinsic aging is caused by environmental factors, principally UVR, smoking, diet, chemicals, trauma, etc. Intrinsic aging occurs within the tissue itself, via reductions in dermal mast cells, fibroblasts, collagen production, flattening of dermal-epidermal junction/loss of rete ridges, as well as being caused by how aging in other organs affects the skin. Intrinsic skin aging can be seen in old age and is characterized by pale(r), drier, less elastic skin with fine wrinkles Exogenous factors also contribute to the aging process. Among all the environmental factors, low-grade chronic UVR exposure contributes to over 80% of facial skin aging. Pollution and smoking can also accelerate the natural aging process. The characteristics of extrinsic skin aging include coarse wrinkling, rough texture, sallow complexion with mottled pigmentation, and loss of skin elasticity.

The mechanism of wrinkle formation is not well understood, but it is generally related to the damage and atrophy of the connective tissues, reduced number of fibroblasts, and their synthetic abilities. The connective tissue is also known as dermal extracellular matrix (ECM), which mainly includes collagen, elastin, and glycosaminoglycans (GAGs). The generation of wrinkles is modulated by intrinsic and extrinsic factors, in which exposure to UVR plays a significant role. UVR increases the degradation of collagen and elastic fibers, causes DNA damage, and induces inflammation, resulting in loss of skin elasticity and formation of wrinkles.

The currently approved and available treatments to correct facial wrinkles and folds include injection of dermal fillers within or beneath the skin. Most dermal fillers are made of materials that can be absorbed by the body over time. However, the dermal fillers only provide a rather temporary effect in filling in wrinkles. Also there are many side effects associated with filler injection include bruising, swelling, pain, itching, infections, lumps, and bumps, change in pigmentation, as well as allergic reactions. Therefore, other strategies to reduce the wrinkles and improve the skin quality are needed to provide better effects and more sustained outcomes, while with less side effects.

MSC have great potential for wrinkle reduction. MSC are precursor cells of fibroblasts, thus MSC itself can directly replenish the lost fibroblasts in the aged skin, restore the infrastructure of skin layers and components that are related to the fibroblasts, including dermal thickness, the density of collagen fibers and other ECM factors. Other mechanisms of MSC in anti-aging and anti-wrinkle on skin are through inhibiting oxidative stress, reducing DNA damage, as well as ameliorating inflammation. MSC can also secrete a variety of cytokines and growth factors that have anti-fibrotic properties, therefore can be used to reduce the excessive fibrotic tissue in the scar.

Therefore the object of the present invention is to provide the compositions of MSC formulation, a characterization method to define the potency of MSC for cosmetic purposes and methods to use the MSC product to ameliorate the skin wrinkles, reduce the signs of aging, attenuate skin scar, prevent hair loss and improve the quality of skin.

### SUMMARY

This invention relates to formulations of mesenchymal stem cells (MSC), a characterization method to define the potency of MSC for cosmetics and use of the MSC formulation to ameliorate the skin wrinkles, reduce the signs of aging and improve the quality of skin. More particularly, the composition of the MSC product formulation includes a solvent and MSC, wherein the solvent comprises three components: a balanced salt solution, an anticoagulant and a human serum albumin. The MSC in the said formulation has the ability to secret at least 1 μg/10⁶ cells/24 hours total collagen or 100 ng/10⁶ cells/24 hours Type I collagen. The methods to use the MSC product for cosmetic include the injection of the MSC formulation into certain layer(s) of the skin (eg. the dermis and/or subcutaneous tissue) using syringe and needles or other delivery devices.

Among the aging-related changes, skin wrinkles are one of the most cosmetically concerning signs of aging for women and men. To correct facial wrinkles and folds, such as nasolabial folds, the currently available treatments include injection of dermal fillers, autologous fat, and autologous fibroblasts within or beneath the skin. Injectable dermal fillers are regulated by the FDA as medical devices. Most dermal fillers are made of materials that can be absorbed by the body over time and provide a temporary effect in filling in wrinkles. These materials include hyaluronic acid, calcium hydroxylapatite, and poly-L-lactic acid (PLLA). The side effects associated with filler injection include bruising, swelling, pain, itching, infections, lumps, and bumps, change in pigmentation, as well as allergic reactions that may lead to a severe reaction. Autologous fat or fibroblasts injection has the potential to avoid hypersensitivity reactions associated with dermal fillers and result in a sustained therapeutic effect. However, these two methods both require surgical procedure to remove tissues from the body.

Mesenchymal stem cells (MSC) are adult stem cells originated from mesoderm. MSC are promising candidate to ameliorate the skin wrinkles, reduce the signs of aging and improve the quality of skin due to their potential of inhibiting oxidative stress, reducing DNA damage, protecting dermal fibroblasts and ECM, as well as ameliorating inflammation. Compared to the current dermal fillers, the MSC are more natural and may provide more components that are beneficial to the skin anti-aging. In another aspect, the use of allogenic MSC does not require the surgical procedure to remove autologous tissue for the cells preparation as compared to autologous fat or fibroblasts injection. Therefore, the use of allogenic MSC for skin anti-aging (eg. anti-wrinkles) purposes is reasonable and has great potential to achieve better outcomes.

To achieve the optimal outcomes on the skin anti-aging, the MSC have to be resuspended in a solvent for the local administration to the skin, and more importantly to preserve the viability and biological functions of the living MSC before administration. After formulation, there are several tests need to be done immediately (quality control) to release a fresh MSC product. Also the released product usually needs to be shipped from manufacturing facility to clinical sites for administration. Thus it is important to find out an optimal composition of the solvent to maintain the quality of MSC during the storage.

In several embodiments, there are provided compositions of the solvent comprising a type of balanced salt solution supplemented with other excipients that are important to maintain the viability and functions of living MSC. Also provided are methods of preparing a MSC product by resuspending the MSC in the solvent and filling the resuspended MSC-solvent mixture into a sterile bottle, and use of the MSC product for injection to the certain layers of the skin.

In several embodiments, the MSC formulation comprise at least three components: a type of balanced salt solution, an anticoagulant and human serum albumin, in addition to MSC.

In several embodiments, the MSC in the said formulation must have the ability to secret at least 10 pg Type I collagen per 1 million (10⁶) cells, when cultured in an in vitro condition.

In several embodiments, the balanced salt solution is, but not limited to, lactated Ringer's solution, multiple electrolyte injection such as Plasma-Lyte A or 0.9% saline (sodium chloride).

In several embodiments, the anticoagulant can be low molecular weight heparin (LMWH) or Ethylenediaminetetraacetic acid (EDTA).

In several embodiments, there are provided methods of preparing a finished MSC pharmaceutical product by resuspending the MSC in the solvent with said compositions and filling the resuspended MSC-solvent mixture into a sterile bottle or vial.

The MSC tissue sources comprise preferably human umbilical cord tissues, which are free of and distinct from umbilical cord blood and comprise the complete umbilical cord solid tissues without removing any solid components including the amniotic epithelium, blood vessels (two arteries and one vein), and Wharton's Jelly stroma of the tissues.

Other examples of the MSC tissue sources include, but are not limited to, bone marrow, adipose tissues, blood, amniotic fluid, dental pulp and placenta.

In several embodiments, the concentration of MSC in the MSC formulations is between 1×10⁶ to 1×10⁷ cells per milliliter.

In several embodiments, the MSC formulation may be used to ameliorate the skin wrinkles, reduce the signs of skin aging, attenuate the skin scar or improve the quality of skin in any part of a body.

In several embodiments, the MSC formulation may be used to prevent the hair loss or promote hair regrowth in any part of a body.

In several embodiments, the MSC formulations are delivered to the skin by serial puncture, tunneling, anterograde, fanning or cross-hatching injection method to the dermal layer or subcutaneous tissue at between 1×10⁵ to 1×10⁷ cells per injection site.

In several embodiments, the devices used for the local delivery of MSC formulations to the skin are medical grade needles with a size from 25 gauge to 32 gauge that are attached to a sterile syringe.

### BRIEF DESCRIPTION OF THE DRAWINGS

FIG. 1 shows the assessment of the dose accuracy. Provided is the table of a cell count test using trypan blue exclusion method was performed to compare the samples with different final concentrations (i.e., 2, 4, or 6×10⁶ cells/mL) at four time points (i.e., 0 hours, 24 hours, 36 hours, and 48 hours). The results showed that the cell concentrations of mesenchymal stem cells (MSC) of all the tested groups were maintained at the 2, 4, or 6×10⁶ cells/mL “±” 10% for up to 48 hours post formulation.

FIGS. 2A-2B show formulated human umbilical cord MSC (hUC-MSC) final products at different concentrations were tested at four-time points to evaluate the dose accuracy and viability via the trypan blue exclusion method. Provided is (A) the graph indicating formulated hUC-MSC products at the selected concentrations maintained the dose accuracy without significant difference for up to 48 hours post the formulation. The dashed line represents the designated cell concentrations (i.e., 2, 4, or 6×10⁶ cells/mL), and the gray-colored area represents the acceptable range (±10%). Data are mean±SEM. 2M/mL represents 2×10⁶ cells/mL, 4M/mL represents 4×10⁶ cells/mL, and 6M/mL represents 6×10⁶ cells/mL. Provided is (A) the graph indicating all the tested groups (i.e., 2, 4, or 6 × 10⁶ cells/mL) met the criteria at all tested points without significant difference. Data are mean±SEM. 2M/mL represents 2×10⁶ cells/mL, 4M/mL represents 4×10⁶ cells/mL, and 6M/mL represents 6×10⁶ cells/mL.

FIG. 3 shows the cell viability results measured by flow cytometry over time to compare those three different final concentrations (i.e., 2, 4, or 6×10⁶ cells/mL). The results showed the formulated hUC-MSC products under all the tested concentrations could maintain the hUC-MSC viability over 90% for up to 48 hours post formulation. The tested samples' cell viability was also evaluated via the trypan blue exclusion method, whose results were consistent with those measured by flow cytometry.

FIG. 4 shows the data of the hUC-MSC identity/purity assay. The surface marker assay was performed over time for the tested samples (i.e., 2, 4, or 6×10⁶ cells/mL). The value (%) of each marker fulfilled the acceptance criteria for hUC-MSC formulated in any of the selected concentrations at all tested time points (i.e., hUC-MSC products showed ≥95% for CD105, CD90, CD73, and ≤2% for CD45, CD34, HLA-DR), which indicated that the formulated hUC-MSC products could preserve their identity property for up to 48 hours even if the cell concentration increased to 6×10⁶ cells/mL.

FIG. 5 shows the results of the tested samples' cell viability via the trypan blue exclusion method, among the stability data of final product lots, whose results were consistent with those measured by flow cytometry.

FIG. 6 shows the stability data of final product lots. All hUC-MSC products (n=3) formulated at 4×10⁶ cells/mL were able to maintain dose accuracy and viability>90% up to 48 hours. A cell count test was performed to compare the samples at four time points (i.e., 0 hours, 24 hours, 36, and 48 hours) to assess the dose accuracy. The results showed that the cell concentration of all the tested groups was maintained at the 4×10⁶ cells/mL “±” 10% for up to 48 hours post formulation, which proved that hUC-MSC products could maintain the dose accuracy for up to 48 hours.

FIG. 6 also presents the cell viability assessment measured by flow cytometry over time. The results showed the formulated hUC-MSC products (n=3) could retain the hUC-MSC viability over 90% for up to 48 hours post formulation.

FIG. 6 also shows that hUC-MSC formulated at 4×10⁶ cells/mL demonstrated a qualified purity/identity property up to 48 hours without any contamination. In order to assess the hUC-MSC identity/purity, the surface marker assay was performed over time for the tested samples. The value (%) of each marker fulfilled the acceptance criteria for hUC-MSC products formulated at 4×10⁶/mL for each tested time point (i.e., hUC-MSC products showed ≥95% for CD105, CD90, CD73, and ≤2% for CD45, CD34, HLA-DR), which indicated that the formulated hUC-MSC products could preserve their identity property for up to 48 hours. Moreover, a passing result (no growth, negative) was obtained for BACT/ALERT incubation (14-Day) for each tested sample during this stability test, demonstrating the sterility of the hUC-MSC products.

FIG. 7 shows the compiled data for the cell count and viability data from the trypan blue exclusion method and the flow cytometry method after final products being transferred to the delivery device(s), indicating that the hUC-MSC products formulated at the representative concentration were able to maintain their stability after being transferred to the delivery device(s) 24±2 hours post formulation.

FIG. 8 shows the mean value from the Surface Phenotype Test performed after final products being transferred to the delivery device(s). The average value of the duplicates indicated that the acceptance criteria was met for the negative-stained CD45, HLA-DR, and CD34 populations, as well as the positive-stained CD90, CD105, and CD73, indicating that the hUC-MSC products formulated at the representative concentration were able to maintain their stability after being transferred to the delivery device(s) 24±2 hours post formulation.

FIG. 9 shows the sterility results using the BacT/ALERT 3D System, after final products being transferred to the delivery device(s). Upon the 14-Day incubation, all samples showed negative test results, indicating that the hUC-MSC products formulated at the representative concentration were able to maintain their stability after being transferred to the delivery device(s) 24±2 hours post formulation.

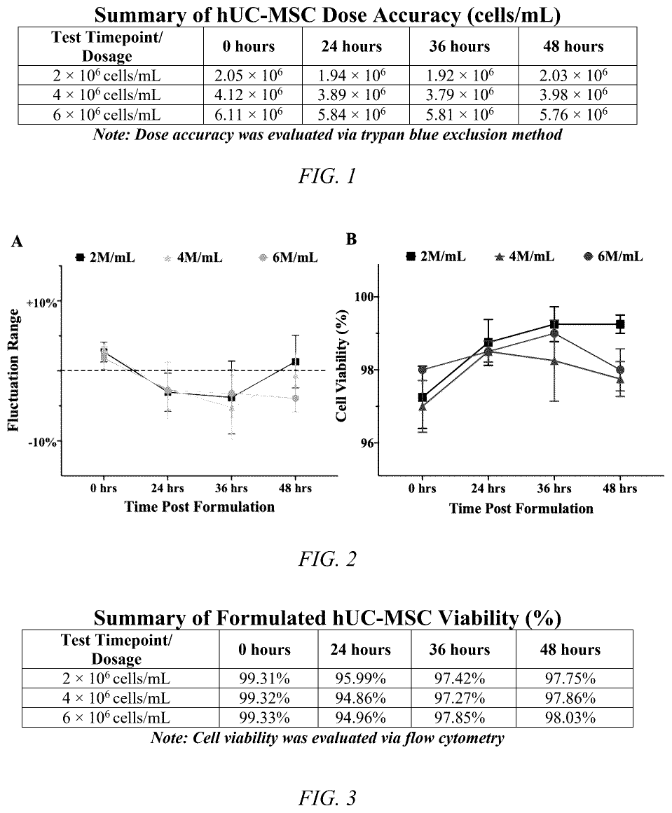

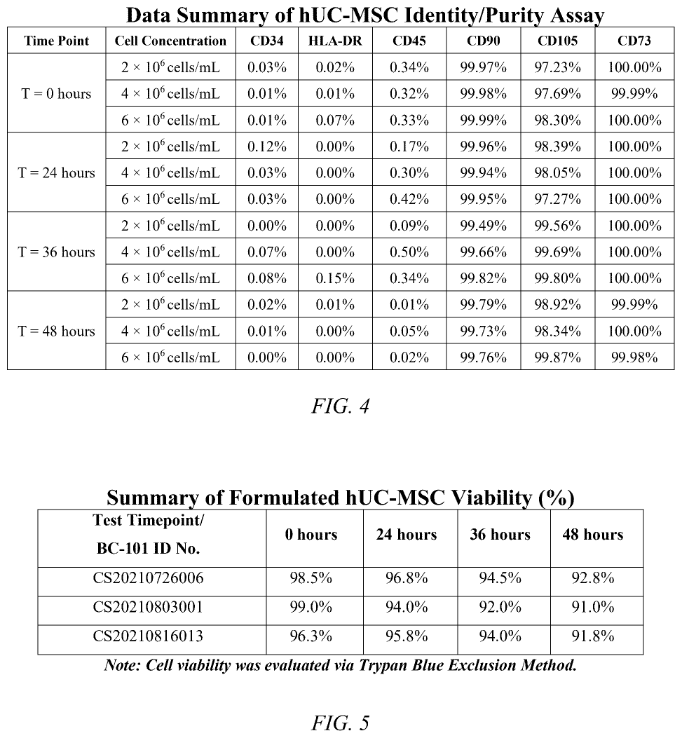

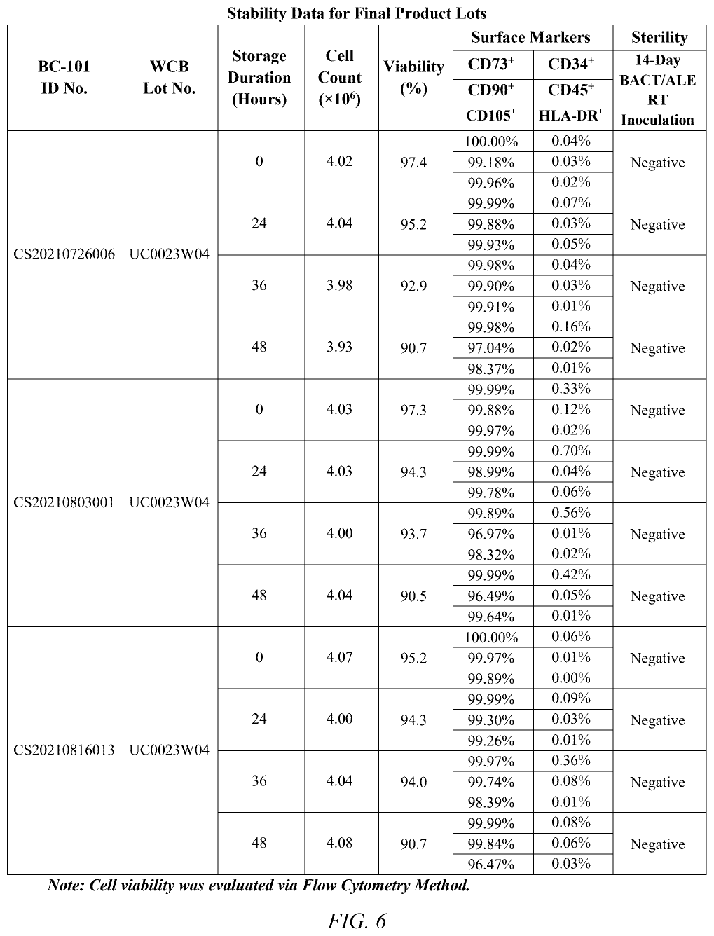

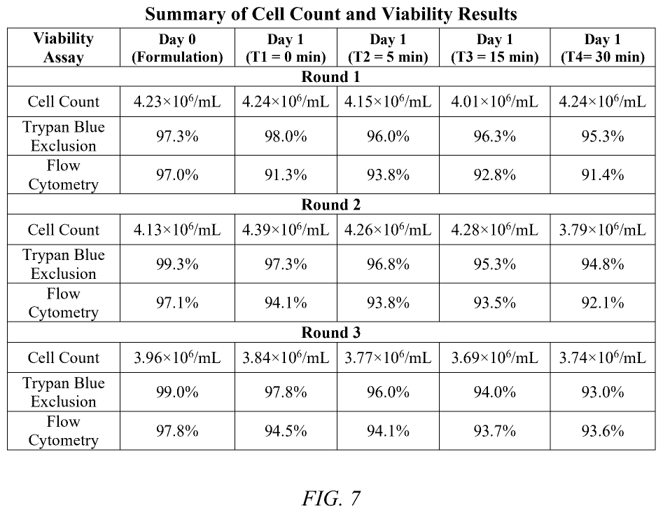

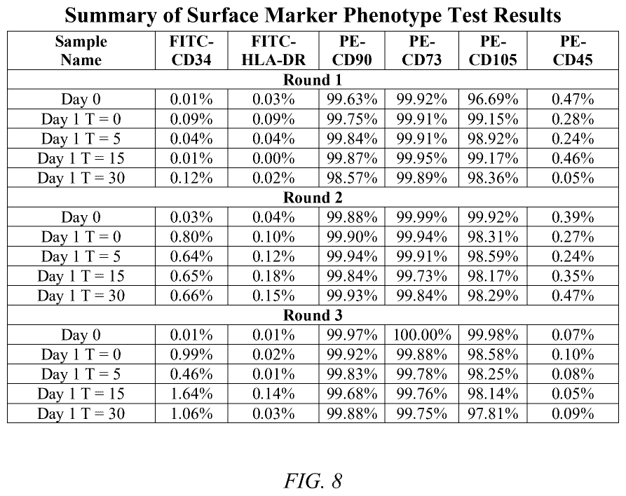

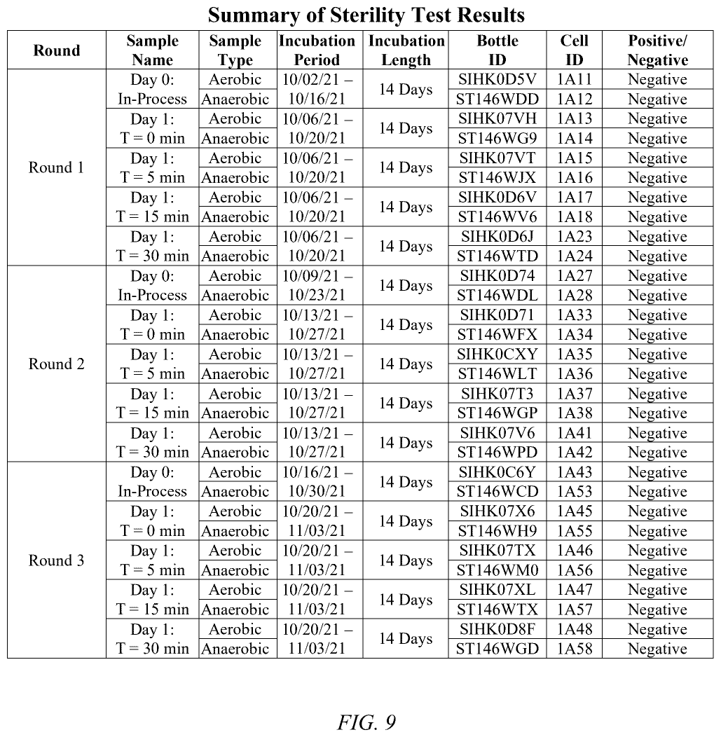

### DETAILED DESCRIPTION

This invention relates to compositions formulations of mesenchymal stem cells (MSC), and use of the MSC product to ameliorate the skin wrinkles, reduce the signs of aging and improve the quality of skin. More particularly, the composition of the MSC product formulation includes a solvent and MSC, wherein the solvent comprises three components: a balanced salt solution, an anticoagulant and a human serum albumin. The MSC in the said formulation has the ability to secret at least 1 μg/10⁶ cells/24 hours total collagen or 100 ng/10⁶cells/24 hours Type I collagen. In other embodiments, this invention also provides the methods to use the MSC product for cosmetic purposes by injection of the MSC formulation into certain layer(s) of the skin (eg. the dermis and/or subcutaneous tissue) using syringe and needles or other delivery devices.

The phrase “Solvent” as used herein means a pharmaceutically-acceptable liquid comprising a solvent with or without supplementing additional excipient or material in carrying or transporting the active components (in this invention MSC), in the finished pharmaceutical products from one organ, or portion of the body, to another organ, or portion of the body. Each carrier must be “acceptable” in the sense of being compatible with the other components or ingredients of the formulation of the finished pharmaceutical products and not injurious to the recipient.

The solvent of this invention is used to resuspend the MSC and maintain the single cell suspension status, viability and biological functions during storage or shipping before administration to recipient(s). MSC are able to secret many adhesive molecules known as extracellular matrix and tend to stick together and form clumps if placed in a steady condition for a prolonged period. Therefore, the solvent in our invention comprise at least three components including a balanced salt solution, an anticoagulant and human serum albumin to mitigate or prevent the aggregation of MSC and loss of viability and biological functions in an appropriate temperature.

A balanced salt solution is a solution made to a

physiological pH and isotonic salt concentration. In several embodiments, the balanced salt solution is an isotonic solution which maintains pH and osmotic balance as well as provides cells with water and essential electrolytes. The balanced salt solution in this invention preferably is an injectable grade solution. Examples include: 1) PLASMA-LYTE A Injection pH 7.4 (Multiple Electrolytes Injection, Type 1, USP); 2) 5% Dextrose and Electrolyte No. 48Injection (Multiple Electrolytes and Dextrose Injection, Type 1, USP); 3) 0.9% Sodium Chloride Injection, USP; 4) Lactated Ringer's Injection, USP.

It has been recognized by the applicants that the extent of the formation of cell aggregate may significantly comprise the cell viability during storage, as the cells in the center or inner space of an aggregate may not get adequate access to the nutrients or oxygen, especially in the case of MSC, a type of cells with high tendency to form aggregation while placed in storage for a prolong period. Applicants have realized that the maintained single cell status is important to mitigate the loss of cell viability for MSC product.

Anticoagulant are chemical substances that are commonly used to prevent or reduce coagulation of blood, prolonging the clotting time. In the current case of MSC solvent, an anticoagulant is used to keep MSC from aggregating. The anticoagulant solution in this invention preferably is Low molecular weight heparin or EDTA.

Ethylenediamine tetraacetic acid (EDTA) is a polyprotic acid containing four carboxylic acid groups and two amine groups with lone-pair electrons that chelate calcium and several other metal ions. EDTA has been widely as the anticoagulant of choice for hematological testing because it allows the best preservation of cellular components and morphology of blood cells. In several embodiments, the solvent for MSC product comprises the EDTA at the concentration of 0.1 mg/mL to 1.5 mg/mL. Thus, according to several embodiments, the final concentration of the low molecular weight heparin in the MSC pharmaceutical product is set at 0.1 mg/mL, 0.2 mg/mL. 0.5 mg/mL, 1.0 mg/mL, 1.5 mg/mL or any other concentration between any of these figures.

Low molecular weight heparin is a class of an anticoagulant which consists of only short chains of natural heparin and has an average molecular weight of less than 8000 Da. At least 60% of all chains have a molecular weight less than 8000 Da. Low molecular weight heparin, due to its more predictable anticoagulant effects that natural heparin, are commonly used to prevent blood clots and treatment of venous thromboembolism. The mechanism of action of low molecular weight heparin to inhibit coagulation process is through binding to antithrombin and accelerate its inhibition of activated factor X, a critical protein in the blood coagulation cascade. The anticoagulant effect of low molecular weight heparin occurs both in vivo and in vitro, thus it is also used to prevent blood clotting in the preparation of blood samples. MSC are able to secret many adhesive molecules known as extracellular matrix and tend to stick together and form clumps if placed in a steady condition for a prolonged period. While in many circumstances, MSC products require the cells in a single cell status (i.e., not in aggregation or in the form of cell clumps) for optimal therapeutic effects. Applicants have found heparin are also able to inhibit the aggregation of MSC in vitro, although the mechanism of action for heparin's inhibition effect on MSC is not clear. The mechanism of action for heparin's inhibition effect on MSC aggregation is presumably different to that on blood coagulation due to the absence of blood coagulation cascade components in MSC suspension in vitro. There are several low molecular weight heparin types due to the different salts, several examples include: Dalteparin sodium, Enoxaparin sodium, and Nadroparin calcium.

Heparin activity (concentration) is measured in either International Units (IU) defined by the World Health Organization (WHO) International Standard, or United States Pharmacopeia (USP) units. There is a small (7-10%) difference between the IU and USP unit. In several embodiments, the solvent for MSC product comprises the low molecular weight heparin at the concentration of 1 IU/mL to 50 IU/mL. Thus, according to several embodiments, the final concentration of the low molecular weight heparin in the MSC pharmaceutical product is set at 1 IU/mL, 2 IU/mL, 5 IU/mL, 10 IU/mL, 20 IU/mL, 30 IU/mL, 40 IU/mL, 50 IU/mL or any other concentration between any of these figures.

The solvent for MSC product comprises the human serum albumin at the concentration of 0.5% to 2% (v/v.). The final concentration of human serum albumin in the MSC pharmaceutical product is set at 0.5%, 1%, 1.5%, 2.0% or any other concentration between any of these figures.

Provided herein are also methods for preparing a finished MSC pharmaceutical product by resuspending the MSC in the solvent with said compositions and filling the resuspended MSC-solvent mixture into a sterile bottle or vial. Several examples of the sterile bottle for a MSC pharmaceutical product include: 5 mL Sterile Vials (SV520), 2 mL Sterile Vials (SV213) from EZVIALZ.

The MSC tissue sources comprise preferably human umbilical cord tissues, which are free of and distinct from umbilical cord blood and comprise the complete umbilical cord solid tissues without removing any solid components including the amniotic epithelium, blood vessels (two arteries and one vein), and Wharton's Jelly stroma of the tissues.

In several embodiments, the MSC containing tissues may be taken from human bone marrow, adipose tissues, blood, amniotic fluid, dental pulp and placenta or other tissues that MSC can be isolated.

In several embodiments, the concentration of MSC in the MSC formulations is between 1×10⁶ to 1×10⁷ cells per milliliter. The final concentration of MSC in the pharmaceutical product is set at 1×10⁶, 2×10⁶, 3×10⁶, 4×10⁶, 5×10⁶, 6×10⁶, 7×10⁶, 8×10⁶, 9×10⁶, 1×10⁷ cells per milliliter or any other concentration between any of these figures.

In several embodiments, the MSC formulation has the ability to secret at least 1 μg/10⁶ cells/24 hours total collagen or 100 ng/10⁶ cells/24 hours Type I collagen. Dermal extracellular matrix (ECM) ensures an essential role in skin cohesion. Among the different kinds of extracellular matrix proteins, collagen is the most abundant type of ECM in the skin. There are many types of collagens. In adults, type I collagen constitutes approximately 80% of dermal collagen. Type 1 collagen loss in the dermis is one of the primary causes of wrinkles seen in aged skin Therefore, supplementation of collagen I is a reasonable and effective strategy to reduce dermal wrinkles. In this current invention, we define the potency of MSC for the wrinkle reduction effects by evaluating their secreting ability of Type I collagen in the culture. The quantitation of secreted type I collagen is analyzed by ELISA of the supernatant from an in vitro culture of MSC.

In several embodiments, the MSC formulation may be used to ameliorate the skin wrinkles, reduce the signs of skin aging, reduce the skin scar and improve the quality of skin in any part of a body.

In one embodiment, the MSC formulation is used to reduce one or more signs of skin aging. Skin aging is defined as the structural, biochemical, and functional changes to the skin as aging, it is characterized by features such as fine lines, wrinkles, loss of elasticity, laxity, age spots, splotches and pigmentation problems, broken capillaries, dull skin texture and color, and other skin flaws Skin aging is a complex biological process influenced by the combination of intrinsic and extrinsic factors, which lead to structural and physiological changes in each skin layer, resulting in changes in skin appearance. Intrinsic aging is generally under genetic and hormonal influence and extrinsic aging is caused by environmental factors, principally UVR, smoking, diet, chemicals, trauma, etc. Intrinsic aging occurs within the tissue itself, via reductions in dermal mast cells, fibroblasts, collagen production, flattening of dermal-epidermal junction/loss of rete ridges, as well as being caused by how aging in other organs affects the skin. Intrinsic skin aging can be seen in old age and is characterized by pale(r), drier, less elastic skin with fine wrinkles. Exogenous factors also contribute to the aging process. Among all the environmental factors, low-grade chronic UVR exposure contributes to over 80% of facial skin aging. Pollution and smoking can also accelerate the natural aging process. The characteristics of extrinsic skin aging include coarse wrinkling, rough texture, sallow complexion with mottled pigmentation, and loss of skin elasticity.

In another embodiment, the MSC formulation is used to ameliorate the skin wrinkles, preferably on the face. A used herein, “wrinkles” are defined as creases, folds, of ridges in the skin. The mechanism of wrinkle formation is not well understood, but it is generally related to the damage and atrophy of the connective tissues, reduced number of fibroblasts, and their synthetic abilities. The connective tissue is also known as dermal extracellular matrix (ECM), which mainly includes collagen, elastin, and glycosaminoglycans (GAGs). The generation of wrinkles is modulated by intrinsic and extrinsic factors, in which exposure to UVR plays a significant role. UVR increases the degradation of collagen and elastic fibers, causes DNA damage, and induces inflammation, resulting in loss of skin elasticity and formation of wrinkles.

In another embodiment, the MSC formulation is used to attenuate scar on the skin. A “scar” is a mark remaining on the skin or within body tissue where a wound, burn, or sore has not healed completely and fibrous connective tissue has developed. Scar tissue has different compositions to the normal tissue that it replaces. Instead of a random basketweave formation of the collagen fibers found in normal tissue, the collagen cross-links and forms a pronounced alignment in a single direction in the scar. Therefore, the scar tissue is usually of inferior functional quality to the normal tissue. For example, scars in the skin are less resistant to ultraviolet radiation, and sweat glands and hair follicles do not grow back within scar tissues.

In another embodiment, the MSC formulation may be used to prevent the hair loss or promote hair regrowth, preferably on the scalp. As used herein, “Hair loss”, also called “Alopecia” is defined as the thinning of hair or loss of hair from part of the head or the body.

In some embodiments, the MSC formulations are delivered to the skin locally by serial puncture, tunneling, anterograde, fanning or cross-hatching injection method to the dermal layer or subcutaneous tissue at between 1×10⁵ to 1×10⁷ cells per injection site. The term “locally”, for administration is intended to deliver the stem cell solution to specific site or sites of the body other than the circulatory system. in the current invention, it refers to delivery of MSC formulation into certain layers of the skin, preferably into the dermis and/or subcutaneous tissues of the skin.

What is claimed is:

1. A formulation of mesenchymal stem cells (MSC) for cosmetic use comprising a solvent and MSC.

2. The formulation of claim 1 comprising: a balanced salt solution, an anticoagulant, and a human serum albumin.

3. The formulation of claim 2, wherein the balanced salt solution is a lactated Ringer's solution, a 0.9% saline (sodium chloride), or a multiple electrolyte injection.

4. The formulation of claim 2, wherein the anticoagulant is a low molecular weight heparin or EDTA.

5. The formulation of claim 4, wherein a final concentration of the low molecular weight heparin is between 1 to 50 IU/mL.

6. The formulation of claim 4, wherein a final concentration of the EDTA is between 0.1 mg/mL to 1.5 mg/mL.

7. The formulation of claim 2, wherein a final concentration of the human serum albumin is between 0.5% to 2% (V/V).

8. A method for preparing a MSC formulation comprising:
providing a tissue source for mesenchymal stem cells (MSC);
Isolating MSC from the tissue source
providing a solvent; and
suspending the MSC in the solvent,
wherein the solvent comprises a balanced salt solution, an anticoagulant, and a human serum albumin.

8. The method of claim 8, wherein the tissue source is an umbilical cord tissue, a bone marrow, an adipose tissue, a blood, an amniotic fluid, a dental pulp, or a placenta.

9. The method of claim 8, wherein the MSC formulation has a concentration of between 1×10⁶ to 1×10⁷ cells per milliliter of solution.

10. The method of claim 8, wherein the MSC formulation has the ability to secret at least 1 μg/10⁶ cells/24 hours total collagen or 100 ng/10⁶ cells/24 hours Type I collagen.

11. A method for apply the MSC formulation to a subject comprising:
administering to a subject in need thereof a MSC product,
wherein the MSC product comprises mesenchymal stem cells (MSC) and a solvent; and
wherein the solvent comprises a balanced salt solution, an anticoagulant, and a human serum albumin.

12. The method of claim 11, wherein the method is for cosmetic use to ameliorate skin wrinkles, reduce the signs of skin aging, attenuate skin scar and prevent hair loss.

13. The method of claim 12, wherein the MSC formulation is delivered to the skin locally to the dermal layer or subcutaneous tissue at between 1×10⁵ to 1×10⁷ cells per injection site.
